(19) United States
(12) Patent Application Publication
(10) Pub. No.: US 2025/0095828 A1
(43) Pub. Date: Mar. 20, 2025

(54) MEDICAL IMAGE SEGMENTATION METHOD BASED ON MULTI-SCALE FEATURE FUSION

(71) Applicants: Guizhou Provincial People's Hospital, Guiyang City (CN); Guizhou University, Guiyang City (CN)

(72) Inventors: Rongpin WANG, Guiyang City, Guizhou Province (CN); Bangkang FU, Guiyang City, Guizhou Province (CN); Junjie HE, Guiyang City, Guizhou Province (CN); Yunsong PENG, Guiyang City, Guizhou Province (CN); Xinhuan SUN, Guiyang City, Guizhou Province (CN)

(21) Appl. No.: 18/883,295

(22) Filed: Sep. 12, 2024

(30) Foreign Application Priority Data
Sep. 15, 2023 (CN) 202311190293 .X

(51) Int. Cl. G16H 30/40; G06T 3/40; G06T 7/10; G06V 10/25; G06V 10/44; G06V 10/771

(52) U.S. Cl. CPC G16H 30/40; G06T 3/40; G06T 7/10; G06V 10/25; G06V 10/44; G06V 10/771; G06T 2207/20084

(57) ABSTRACT

Provided is a medical image segmentation method based on multi-scale feature fusion. The method includes acquiring medical image data of a same type, and sketching a region of interest (ROI); preprocessing sketched image data; constructing a multi-scale feature extraction module, a multi-scale feature fusion module, and an encoder-decoder deep learning network model; performing 5-fold cross-validation on the network model; and evaluating a medical image segmentation result output by the model with an evaluation index including a dice, an accuracy, a precision and a recall. This application extracts multi-scale features comprehensively from an encoder, a decoder, and connection between the encoder and the decoder in the network, and effectively learns multi-scale information in the image. The multi-scale features are fused by an interactive module, so this application relieves feature conformity caused by direct multi-scale fusion, thereby improving performance of the medical image segmentation.

### CROSS REFERENCE TO RELATED APPLICATION

This patent application claims the benefit and priority of Chinese Patent Application No. 202311190293.X, filed with the China National Intellectual Property Administration on Sep. 15, 2023, the disclosure of which is incorporated by reference herein in its entirety as part of the present application.

### TECHNICAL FIELD

The present disclosure belongs to the technical field of medical image processing, and particularly relates to a medical image segmentation method based on multi-scale feature fusion.

### BACKGROUND

Medical image segmentation is to segment specific meaningful parts for quantitative or qualitative analysis. It is a crucial issue in medical image analysis, and also an important constituent in such clinical applications as aided diagnosis, aided treatment planning, efficacy evaluation, and prognostic prediction. Multi-scale information in images can describe features richly, and improve the accuracy of segmentation. However, due to complexity of the information, fuzziness of boundaries and different sizes of regions of interest (ROIs), full utilization of the multi-scale information in the medical image segmentation remains a challenge.

With the development of deep learning, convolutional neural networks (CNNs) have been widely applied in the field of medical image segmentation. The CNNs can acquire rich feature information. However, due to local connectivity, translation invariance, and the like, they lack a remote modeling capability, and cannot completely extract global information. In order to overcome shortages of the CNNs in remote modeling, vision transformers (ViTs) are proposed to treat computer vision tasks. The ViTs are desirable to extract the global information for their strong attention mechanism. Nevertheless, with the computational cost far higher than that of the CNNs, the ViTs are usually used only for learning single-scale features. There emerge segmentation networks based on the CNNs and the transformers. Restricted by the computation cost, most of them are realized by extracting multi-scale features from low-resolution high-level semantic features, or learning multi-scale features between an encoder and a decoder. The expensive computational cost not only restricts the comprehensive learning of the multi-scale features, but also impairs ideal fusion of the extracted multi-scale features.

To sum up, the existing medical image segmentation methods have the following problems: The multi-scale information cannot be acquired completely. Restricted by the computational cost, some methods cannot learn the multi-scale information comprehensively. On the other hand, the acquired multi-scale information are only concatenated or added simply, while a difference between information of different scales is not considered. Consequently, features of different scales cannot used fully to affect a final segmentation result.

### SUMMARY

An embodiment of the present disclosure aims to provide a medical image segmentation method based on multi-scale feature fusion, to realize more comprehensive acquisition of multi-scale information and more sufficient fusion of multi-scale features through a lightweight model.

In order to solve the above technical problems, the present disclosure employs the following technical solutions: The present disclosure provides a medical image segmentation method based on multi-scale feature fusion, including the following steps:
S1: acquiring medical image data of a same type, and sketching an ROI;
S2: preprocessing the medical image data;
S3: constructing a multi-scale feature extraction module;
S4: constructing a multi-scale feature fusion module;
S5: constructing an encoder-decoder deep learning network model;
S6: performing 5-fold cross-validation on the network model; and
S7: evaluating segmentation performance with an evaluation index.

Further, the S2 specifically includes:

S21: reading the medical image data acquired in the S1 into a memory, and seeking a mean and a standard deviation of the data:

$$μ = \frac{1}{n} {Σ}_{i = 1}^{n} {x}_{i}$$

$$std = \sqrt{\frac{1}{n} {Σ}_{i = 1}^{n} {( {x}_{i} - μ )}^{2}}$$

S22: performing normalization on an image according to the mean and the standard deviation:

$$Y = \frac{X - μ}{std}$$

where, μ represents the mean, std is the standard deviation, n represents a number of samples, xᵢ represents an ith sample, X represents a sample population, and Y represents a sample population obtained after the normalization; and

S23: preprocessing the data by flipping the image and scaling the image to a same size.

Further, when the multi-scale feature extraction module is constructed in the S3, the multi-scale feature extraction module is constructed in an encoder and a decoder first, and then a cross-feature extraction module is constructed between the encoder and the decoder.

Further, the multi-scale feature extraction module includes a CNN channel based on local information feature extraction and a transformer channel based on global information feature extraction; the CNN channel includes a convolutional layer, a normalization layer, and an activation function; and the CNN channel has a following computing method:

$${Y}_{1} = Relu ( BN ( Conv ( {Y}_{0} ) ) )$$

$${Y}_{2} = Relu ( X + B N ( Conv ( {Y}_{1} ) ) )$$

where, Y₀ represents an input feature map, Conv represents a convolutional operation, BN is a normalization operation, Relu is the activation function, Y₁ is a result subjected to convolution, normalization and the activated function once, and Y₂ is a feature map output finally through the CNN channel; and a self-attention of the transformer channel is computed by:

$$Q , K , V = DewConv ( {Y}_{0} )$$

$${Q}_{1} = {Conv}_{S = r} ( Q )$$

$${K}_{1} = {Conv}_{S = r} ( K )$$

$$Light\_Atten ( Q , K , V ) = Soft max ( \frac{{Q}_{1} {K}_{1}^{T}}{\sqrt{{d}_{k}}} ) V$$

where, DewConv represents depthwise separable convolution, Y₀ represents the input feature map, Q is a query matrix, K is a key matrix, V is a value matrix, Conv_{S=r} represents convolution at a stride of r, the matrix Q and the matrix K are respectively subjected to the convolution to obtain a matrix Q₁ and a matrix K₁, Light_Atten represents a lightweight self-attention, dₖ is a dimension of the key matrix, and Softmax is a mapping function.

Further, when the cross-feature extraction module is constructed, an input of the cross-feature extraction module is a feature map output by the encoder; the encoder includes three layers of multi-scale feature extraction modules and multi-scale feature fusion modules; for the input feature map, a feature map output by a first layer of multi-scale feature extraction modules and multi-scale feature fusion modules is α₁, and a feature map output by a second layer and a feature map output by a third layer are respectively α₂ and α₃; and the cross-feature extraction module is constructed as follows: performing matrix multiplication on the feature map α₁ and the feature map α₂, performing normalization on a result of the matrix multiplication, performing matrix multiplication on a processed result and the feature map α₃, and performing residual connection on a result of the matrix multiplication and the feature map α₃.

Further, the multi-scale feature fusion module in the S4 includes two inputs, specifically a local information feature f₁ extracted by a CNN and a global information feature f₂ extracted by a transformer; for the multi-scale feature fusion module, the f₁ and the f₂ are concatenated into a feature map matrix, the feature map matrix is split into v different portions in a first dimension, a shift operation is performed on h portions along a y-axis direction, and spatial position information is learned with convolution to obtain a feature map Z_{V}; a shift operation is performed in an x-axis direction, and spatial information is learned with the convolution; and residual connection is performed on an obtained feature map Z_{H} and the input feature map matrix:

$${Z}_{V} = Conv ( Roll ( Chunk ( Z , v ) , Vertical ) )$$

$${Z}_{H} = Conv ( Roll ( Chunk ( {Z}_{V} , h ) , Horizontal ) )$$

where, Z is the concatenated feature map matrix, v is a portion to be split, Chunk represents a split operation, Roll represents the shift operation, Conv represents a convolutional operation, Vertical represents the shift operation along the y-axis direction, and Horizontal represents the shift operation along the x-axis direction.

Further, the encoder-decoder deep learning network model in the S5 consists of an encoder and a decoder; the encoder-decoder deep learning network model includes four layers, and each of the four layers includes the multi-scale feature extraction module and the multi-scale feature fusion module; a preprocessed medical image is input, a local feature is generalized through a convolutional module having a stride, and a feature map is downsampled; an obtained input feature map enters a CNN local feature extraction channel and a transformer global feature extraction channel to obtain two feature matrices having a same size; the two feature matrices are concatenated and input to the multi-scale feature fusion module; and the multi-scale feature fusion module is configured to perform interaction on features of different scales through a split operation and a shift operation.

Further, for the features of the different scales obtained by the encoder, attentions of the features are fused through a cross-feature extraction module between the encoder and the decoder; an obtained feature is connected to the decoder; and through extraction, fusion and upsampling on multi-scale features, a segmented mask image is obtained.

Further, the S6 specifically includes: uniformly splitting a dataset into five subsets having a similar size, including four subsets used in training, and one subset used as a validation set; performing validation cyclically five times; and averaging validation results in the five times to obtain a final result.

Further, a model parameter is optimally trained with a weighted cross-entropy loss function and a dice loss function by:

$${L}_{Dice} = 1 - \frac{2 \vert P ⋂ G \vert}{\vert P \vert + \vert G \vert}$$

$${L}_{CE} = - \frac{1}{N} {\sum }_{i = 1}^{C} {\sum }_{j = 1}^{N} {g}_{ij} log {p}_{ij}$$

$${L}_{total} = α {L}_{Dice} + ( 1 - α ) {L}_{CE}$$

where, L_{Dice} represents the dice loss function; L_{CE} represents the cross-entropy loss function; Lₜₒₜₐₗ represents a total loss function; G represents a real label; P represents a segmented region predicted by the network model; N represents a total number of pixels in an input image; in gᵢⱼ, i represents a category of a label, and j represents a pixel, and the gᵢⱼ has a value of 1 when a predicted label of the pixel is the same as the real label, and a value of 0 when the predicted label of the pixel is different from the real label; pᵢⱼ is a probability that the model outputs the pixel j; and α is an artificially set parameter, and is in an interval from 0 to 1.

The present disclosure has following beneficial effects:

From the encoder, the decoder, and the connection between the encoder and the decoder in the network, the present disclosure extracts multi-scale features comprehensively, and learns multi-scale information in the image effectively. After the multi-scale features are extracted, the multi-scale features are fused by an interactive module. This effectively relieves feature conformity caused by direct multi-scale fusion, thereby improving performance of the medical image segmentation.

### BRIEF DESCRIPTION OF THE DRAWINGS

To describe the technical solutions in the embodiments of the present disclosure or in the prior art more clearly, the following briefly describes the accompanying drawings required for describing the embodiments or the prior art. Apparently, the accompanying drawings in the following description show merely some embodiments of the present disclosure, and a person of ordinary skill in the art may still derive other drawings from these accompanying drawings without creative efforts.

FIG. 1 is a schematic view illustrating multi-scale feature extraction in an encoder and a decoder;

FIG. 2 is a schematic view illustrating a cross-feature extraction module;

FIG. 3 is a schematic view illustrating multi-scale feature fusion;

FIG. 4 illustrates an encoder-decoder network model; and

FIG. 5 is a flowchart according to the present disclosure.

### DETAILED DESCRIPTION OF THE EMBODIMENTS

The technical solutions of the embodiments of the present disclosure are clearly and completely described below with reference to the drawings in the embodiments of the present disclosure. Apparently, the described embodiments are merely a part rather than all of the embodiments of the present disclosure. All other embodiments obtained by a person of ordinary skill in the art based on the embodiments of the present disclosure without creative efforts shall fall within the protection scope of the present disclosure.

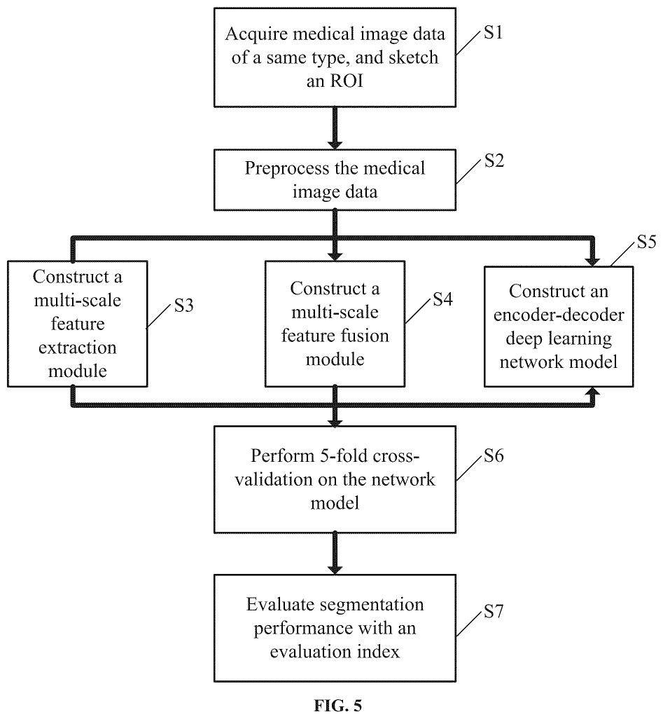

As shown in FIG. 5, an embodiment of the present disclosure provides a medical image segmentation method based on multi-scale feature fusion, including the following steps:

### Embodiment 1

Step 1: Medical image data of a same type is acquired, and an ROI is sketched.

In the embodiment, a common dataset for dermoscopic images is acquired and used. The dataset includes 2,594 images of different sizes and a red, green, and blue (RGB) format. The ROI is sketched by a professional physician.

Step 2: The medical image data is preprocessed.

The images are read into a memory. A mean and a standard deviation of the data are sought. Normalization is performed on an image according to the mean and the standard deviation. The data is processed by flipping the image and scaling the image to a same size. A computing method in the normalization is as follows:

$$μ = \frac{1}{n} {\sum }_{i = 1}^{n} {x}_{i}$$

$$std = \sqrt{\frac{1}{n} {\sum }_{i = 1}^{n} {( {x}_{i} - μ )}^{2}}$$

$$Y = \frac{X - μ}{std}$$

where, μ represents the mean, std is the standard deviation, n represents a number of samples, xᵢ represents an ith sample, X represents a sample population, and Y represents a sample population obtained after the normalization.

Step 3: A multi-scale feature extraction module is constructed.

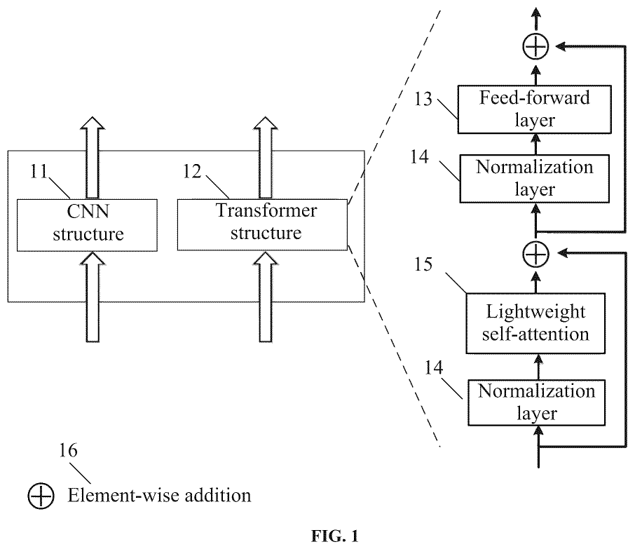

The multi-scale feature extraction module is constructed in an encoder and a decoder. As shown in FIG. 1, the multi-scale feature extraction module includes two channels, specifically a CNN structure 11 based on local information feature extraction, and a transformer structure 12 based on global information feature extraction. An input feature map is parallel input to the two different channels. The CNN is configured to learn detailed information through convolution, while the transformer is configured to compute a lightweight self-attention for remote dependency modeling. The lightweight self-attention 15 is computed by:

$$Q , K , V = DewConv ( {Y}_{0} )$$

$${Q}_{1} = {Conv}_{S = r} ( Q )$$

$${K}_{1} = {Conv}_{S = r} ( K )$$

$$Light\_Atten ( Q , K , V ) = Soft max ( \frac{{Q}_{1} {K}_{1}^{T}}{\sqrt{{d}_{k}}} ) V$$

where, Y₀ represents the input feature map, and DewConv represents depthwise separable convolution composed of depthwise convolution and pointwise convolution. Through the separable convolution, three mapping matrices are obtained, including a query matrix Q, a key matrix K and a value matrixV. Conv_{S=r} represents convolution at a stride of r. The matrix Q and the matrix K are respectively subjected to the convolution to obtain a matrix Q₁ and a matrix K₁. Light_Atten represents the lightweight self-attention. The Softmax function maps an output value to an interval [0,1].

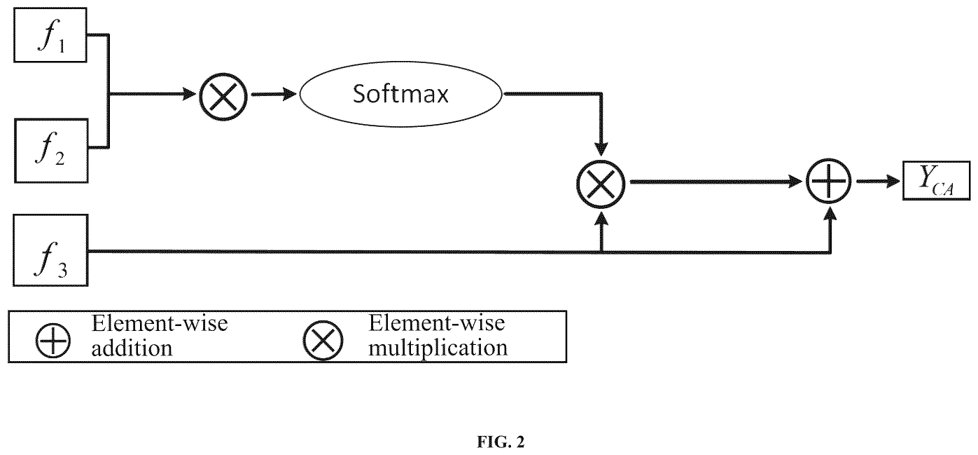

A cross-feature extraction module is constructed between the encoder and the decoder. As shown in FIG. 2, the cross-feature extraction module is structurally different from the multi-scale feature extraction module in the encoder and the decoder. A feature map of a dermoscopic image processed and output by the encoder serves as an input of the cross-feature extraction module. The encoder includes three layers of multi-scale feature extraction modules and multi-scale feature fusion modules. Feature maps output by different layers are respectively represented by α₁, α₂ and α₃. Matrix multiplication is performed on the feature map α₁ and the feature map α₂. Softmax normalization is performed on an obtained result. Matrix multiplication is performed on a processed result and the feature map α₃. Residual connection is performed on a result of the matrix multiplication and the feature map α₃. In this process, cross attentions of feature maps of different scales are computed, thereby facilitating learning of multi-scale features.

Step 4: A multi-scale feature fusion module is constructed.

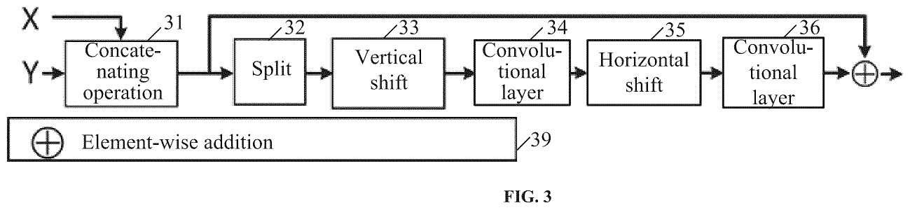

This module can relieve the problem of feature inconformity caused by features of different scales. As shown in FIG. 3, this module includes two inputs, specifically a local information feature f₁ extracted by a CNN and a global information feature f₂ extracted by a transformer. The two inconsistent features are concatenated 31 into a feature map matrix. The feature map matrix is split 32 into v different portions in a first dimension. A shift operation 33 is performed on h portions along a y-axis direction. Spatial position information is learned with convolution. Likewise, a processed feature map is split. A shift operation is performed in an x-axis direction. Spatial information is learned with the convolution. Residual connection is performed on an obtained result and the input feature map matrix. The computing equation is as follows:

$${Z}_{V} = Conv ( Roll ( Chunk ( Z , v ) , Vertical ) )$$

$${Z}_{H} = Conv ( Roll ( Chunk ( {Z}_{V} , h ) , Horizontal ) )$$

where, Z is the concatenated feature map matrix, v is a portion to be split, Chunk represents a split operation, Roll represents the shift operation, Conv represents a convolutional operation, Z_{V} represents a feature map generated in the y-axis direction, and Z_{H} represents a feature map generated in the x-axis direction.

Step 5: An encoder-decoder deep learning network model is constructed.

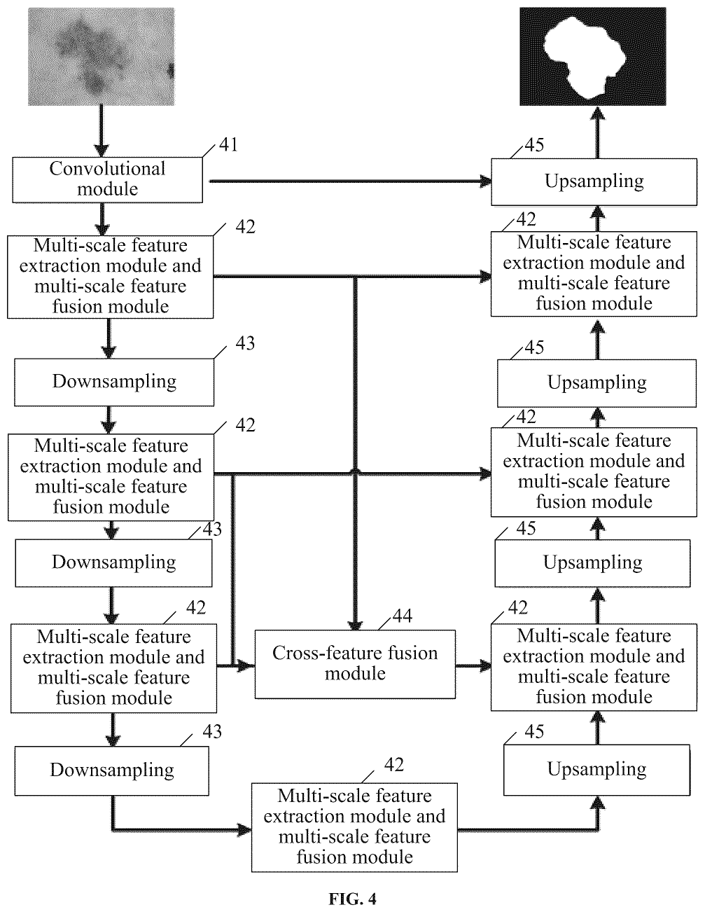

As shown in FIG. 4, the encoder-decoder deep learning network model consists of an encoder and a decoder. The whole encoder-decoder deep learning network model includes four layers, and each of the four layers includes the multi-scale feature extraction module and the multi-scale feature fusion module 42. A preprocessed dermoscopic image is input. A local feature is generalized through a convolutional module 41 having a stride of 1 and a kernel size of 3. A feature map is downsampled 43 at a sampling rate of 2. An obtained input feature map enters a CNN local feature extraction channel and a transformer global feature extraction channel to obtain two feature matrices having a same size. The two feature matrices are concatenated and input to the multi-scale feature fusion module. This module is configured to perform interaction on features of different scales through a split operation and a shift operation.

For the fused feature obtained through the split operation and the shift operation on different layers in the encoder, attentions of the features are interacted through a cross-feature extraction module 42 between the encoder and the decoder. An obtained feature is connected to the decoder. Through extraction, fusion and upsampling 45 on multi-scale features, a segmented mask image is obtained.

Step 6: 5-fold cross-validation is performed on the network model.

In order to reduce a random error caused by random splitting, the 5-fold cross-validation is performed on the network model for training and evaluation. A dataset is uniformly split into five subsets having a similar size. Four subsets are used in training, and one subset is used as a validation set. The validation is performed cyclically five times. Each subset can be taken as the validation set. Validation results in the five times are averaged to obtain a final result.

A model parameter is optimally trained with a weighted cross-entropy loss function and a dice loss function in the network model by:

$${L}_{Dice} = 1 - \frac{2 \vert P ⋂ G \vert}{\vert P \vert + \vert G \vert}$$

$${L}_{CE} = - \frac{1}{N} {\sum }_{i = 1}^{C} {\sum }_{j = 1}^{N} {g}_{ij} log {p}_{ij}$$

$${L}_{total} = α {Loss}_{Dice} + ( 1 - α ) {Loss}_{CE}$$

where, L_{Dice} represents the dice loss function, L_{CE} represents the cross-entropy loss function, and Lₜₒₜₐₗ represents a total loss function. G represents a real label, and P represents a segmented region predicted by the network model. N represents a total number of pixels in an input image. In gᵢⱼ, i represents a category of a label, and j represents a pixel, and the gᵢⱼ has a value of 1 when a predicted label of the pixel is the same as the real label, and a value of 0 when the predicted label of the pixel is different from the real label. pᵢⱼ is a probability that the model outputs the pixel j, and a is an artificially set parameter, and is in an interval from 0 to 1.

Step 7: A medical image segmentation result is evaluated with an evaluation index including a dice, an accuracy, a precision and a recall.

In order to compute the dice, the accuracy, the precision and the recall, a true positive (TP) index, a true negative (TN) index, a false positive (FP) index and a false negative (FN) index of a model segmented result are computed by:

$$TP = {\sum }_{i = 1}^{n} {p}_{i} \cdot {q}_{i}$$

$$TN = {\sum }_{i = 1}^{n} ( 1 - {p}_{i} ) \cdot ( 1 - {q}_{i} )$$

$$FP = {\sum }_{i = 1}^{n} ( 1 - {p}_{i} ) \cdot {q}_{i}$$

$$FN = {\sum }_{i = 1}^{n} {p}_{i} \cdot ( 1 - {q}_{i} )$$

where, pᵢ, qᵢ ∈ {0,1}, 0 represents a negative voxel, 1 represents a positive voxel, pᵢ is a result of an ith voxel predicted by the model, and qᵢ is an annotated result sketched by the professional physician in the step 1. The dice, the accuracy, the precision and the recall are computed by:

$$Dice = \frac{2 \times TP}{2 \times TP + FP + FN}$$

$$Accuracy = \frac{TP + TN}{TP + TN + FP + FN}$$

$$Precision = \frac{TP}{TP + FP}$$

$$Recall = \frac{TP}{TP + FN}$$

Table 1 illustrates comparison between the present disclosure and the existing method in a segmentation result on a dermoscopic image, including the dice, the accuracy, the precision and the recall.

TABLE 1
Comparison between the present disclosure and the
prior art in the medical image segmentation effect

| Method | Dice | Accuracy | Precision | Recall |
|---|---|---|---|---|
| U-Net | 0.8924 | 0.9640 | 0.8898 | 0.8970 |
| UTNet | 0.8936 | 0.9655 | 0.8745 | 0.9148 |
| Swin-Unet | 0.8590 | 0.9462 | 0.9057 | 0.8189 |
| Present disclosure | 0.9109 | 0.9725 | 0.8954 | 0.9281 |

As can be seen from the evaluation results, the method of the present disclosure achieves a desirable segmentation result in the four different evaluation indexes.

Learning on multi-scale information in the medical image segmentation has a great impact on the segmentation result. The segmentation method in the prior art neither acquires the multi-scale information completely, nor fuses the multi-scale features well, thereby affecting the image segmentation result. The method of the present disclosure learns the multi-scale information completely from the encoder, the decoder and the connection between the encoder and the decoder, and fuses the extracted multi-scale features completely. The desirable segmentation performance of the present disclosure is evaluated on medical images of different types.

Each embodiment in this specification is described in a related manner, each embodiment focuses on the difference from other embodiments, and the same and similar parts between the embodiments may refer to each other. For a system embodiment, since it is basically similar to the method embodiment, the description is relatively simple, and reference can be made to the description of the method embodiment.

Any modifications, equivalent substitutions and improvements made within the spirit and scope of the present disclosure should fall within the protection scope of the present disclosure. Any modifications, equivalent substitutions, and improvements made within the spirit and scope of the present disclosure should fall within the protection scope of the present disclosure.

1. A medical image segmentation method based on multi-scale feature fusion, comprising the steps:
S1: acquiring medical image data of a same type, and sketching a region of interest (ROI);
S2: preprocessing the medical image data obtained in S1;
S3: constructing a multi-scale feature extraction module;
S4: constructing a multi-scale feature fusion module;
S5: constructing an encoder-decoder deep learning network model;
S6: performing 5-fold cross-validation on the network model; and
S7: evaluating segmentation performance with an evaluation index.

2. The medical image segmentation method based on multi-scale feature fusion according to claim 1, wherein the S2 preprocessing the medical image date comprises:
S21: reading the medical image data acquired in S1 into a memory, and seeking a mean and a standard deviation of the data:
$$μ = \frac{1}{n} {\sum }_{i = 1}^{n} {x}_{i}$$
$$std = \sqrt{\frac{1}{n} {\sum }_{i = 1}^{n} {( {x}_{i} - μ )}^{2}}$$
S22: performing normalization on an image according to the mean and the standard deviation:
$$Y = \frac{X - μ}{std}$$
wherein, μ represents the mean, std is the standard deviation, n represents a number of samples, xᵢ represents an ith sample, X represents a sample population, and Y represents a sample population obtained after the normalization; and
S23: preprocessing the data by flipping the image and scaling the image to a same size.

3. The medical image segmentation method based on multi-scale feature fusion according to claim 1, wherein when the multi-scale feature extraction module is constructed in S3, the multi-scale feature extraction module is constructed in an encoder and a decoder first, and then a cross-feature extraction module is constructed between the encoder and the decoder.

4. The medical image segmentation method based on multi-scale feature fusion according to claim 3, wherein the multi-scale feature extraction module comprises a convolutional neural network (CNN) channel based on local information feature extraction and a transformer channel based on global information feature extraction; the CNN channel comprises a convolutional layer, a normalization layer and an activation function; and the CNN channel has a following computing method:
$${Y}_{1} = Re lu ( BN ( Conv ( {Y}_{0} ) ) )$$
$${Y}_{2} = Re lu ( X + BN ( Conv ( {Y}_{1} ) ) )$$
wherein, Y₀ represents an input feature map, Conv represents a convolutional operation, BN is a normalization operation, Relu is the activation function, Y₁ is a result subjected to convolution, normalization and the activated function once, and Y₂ is a feature map output finally through the CNN channel; and a self-attention of the transformer channel is computed by:
$$Q , K , V = DewConv ( {Y}_{0} )$$
$${Q}_{1} = {Conv}_{S = r} ( Q )$$
$${K}_{1} = {Conv}_{S = r} ( K )$$
$$Light\_Atten ( Q , K , V ) = Soft max ( \frac{{Q}_{1} {K}_{1}^{T}}{\sqrt{{d}_{k}}} ) V$$
wherein, DewConv represents depthwise separable convolution, Y₀ represents the input feature map, Q is a query matrix, K is a key matrix, V is a value matrix, Conv_{S=r} represents convolution at a stride of r, the matrix Q and the matrix K are respectively subjected to the convolution to obtain a matrix Q₁ and a matrix K₁, Light_Atten represents a lightweight self-attention, dₖ is a dimension of the key matrix, and Softmax is a mapping function.

5. The medical image segmentation method based on multi-scale feature fusion according to claim 3, wherein when the cross-feature extraction module is constructed, an input of the cross-feature extraction module is a feature map output by the encoder; the encoder comprises three layers of multi-scale feature extraction modules and multi-scale feature fusion modules; for the input feature map, a feature map output by a first layer of multi-scale feature extraction modules and multi-scale feature fusion modules is α₁, and a feature map output by a second layer and a feature map output by a third layer are respectively α₂, and α₃; and the cross-feature extraction module is constructed as follows:
performing matrix multiplication on the feature map α₁ and the feature map α₂, performing normalization on a result of the matrix multiplication;
performing matrix multiplication on a processed result and the feature map α₃; and
performing residual connection on a result of the matrix multiplication and the feature map α₃.

6. The medical image segmentation method based on multi-scale feature fusion according to claim 1, wherein the multi-scale feature fusion module in S4 comprises two inputs, a local information feature f₁ extracted by a CNN and a global information feature f₂ extracted by a transformer;
for the multi-scale feature fusion module, the f₁ and the f₂ and the/are concatenated into a feature map matrix, the feature map matrix is split into v different portions in a first dimension, a shift operation is performed on h portions along a y-axis direction, and spatial position information is learned with convolution to obtain a feature map Z_{V} a shift operation is performed in an x-axis direction, and spatial information is learned with the convolution; and residual connection is performed on an obtained feature map Z_{H}, and the input feature map matrix:
$${Z}_{V} = Conv ( Roll ( Chunk ( Z , v ) , Vertical ) )$$
$${Z}_{H} = Conv ( Roll ( Chunk ( {Z}_{V} , h ) , Horizontal ) )$$
wherein, Z is the concatenated feature map matrix, v is a portion to be split, Chunk represents a split operation, Roll represents the shift operation, Conv represents a convolutional operation, Vertical represents the shift operation along the y-axis direction, and Horizontal represents the shift operation along the x-axis direction.

7. The medical image segmentation method based on multi-scale feature fusion according to claim 1, wherein the encoder-decoder deep learning network model in S5 consists of an encoder and a decoder;
the encoder-decoder deep learning network model comprises four layers, and each of the four layers comprises the multi-scale feature extraction module and the multi-scale feature fusion module;
a preprocessed medical image is input, a local feature is generalized through a convolutional module having a stride, and a feature map is downsampled;
an obtained feature map enters a CNN local feature extraction channel and a transformer global feature extraction channel to obtain two feature matrices having a same size;
the two feature matrices are concatenated and input to the multi-scale feature fusion module; and
the multi-scale feature fusion module is configured to perform interaction on features of different scales through a split operation and a shift operation.

8. The medical image segmentation method based on multi-scale feature fusion according to claim 7, wherein for the features of the different scales obtained by the encoder, attentions of the features are fused through a cross-feature extraction module between the encoder and the decoder;
an obtained feature is connected to the decoder; and
through extraction, fusion and upsampling on multi-scale features, a segmented mask image is obtained.

9. The medical image segmentation method based on multi-scale feature fusion according to claim 1, wherein S6 comprises: uniformly splitting a dataset into five subsets having a similar size, comprising four subsets used in training, and one subset used as a validation set;
performing validation cyclically five times; and
averaging validation results in the five times to obtain a final result.

10. The medical image segmentation method based on multi-scale feature fusion according to claim 9, wherein a model parameter is optimally trained with a weighted cross-entropy loss function and a dice loss function by:
$${L}_{Dice} = 1 - \frac{2 \vert P ⋂ G \vert}{\vert P \vert + \vert G \vert}$$
$${L}_{CE} = - \frac{1}{N} {\sum }_{i = 1}^{C} {\sum }_{j = 1}^{N} {g}_{ij} log {p}_{ij}$$
$${L}_{total} = α {L}_{Dice} + ( 1 - α ) {L}_{CE}$$
wherein, L_{Dice} represents the dice loss function; L_{CE} represents the cross-entropy loss function; Lₜₒₜₐₗ represents a total loss function, G represents a real label; P represents a segmented region predicted by the network model; N represents a total number of pixels in an input image; in gᵢⱼ, i represents a category of a label, and j represents a pixel, and the gᵢⱼ has a value of 1 when a predicted label of the pixel is the same as the real label, and a value of 0 when the predicted label of the pixel is different from the real label; pᵢⱼ is a probability that the model outputs the pixel j; and α is an artificially set parameter, and is in an interval from 0 to 1.
